(19) United States
(12) Patent Application Publication
(10) Pub. No.: US 2008/0249143 A1
(43) Pub. Date: Oct. 9, 2008

(54) NEW FORMULATIONS AND USE THEREOF

(76) Inventors: Fredrik Nicklasson, Helsingborg (SE); Kristina Thyresson, Helsingborg (SE); Nils-Olof Lindberg, Helsingborg (SE); Alice C. Martino, Kalamazoo, MI (US); Katarina Lindell, Helsingborg (SE)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 12/143,001

(22) Filed: Jun. 20, 2008

Related U.S. Application Data
(63) Continuation of application No. 10/816,672, filed on Apr. 2, 2004.
(60) Provisional application No. 60/460,253, filed on Apr. 3, 2003.

(30) Foreign Application Priority Data
Mar. 26, 2003 (SE) 0300831-5

(51) Int. Cl. A61K 31/4402

(52) U.S. Cl. 514/357

(57) ABSTRACT

A pharmaceutical formulation comprising one or more active pharmaceutical ingredients (APIs) suitable for intraoral uptake, that comprises cocoa powder, a process for manufacturing the formulation and use of the formulation for medical treatment.

This application hereby claims priority from U.S. provisional application 60/460,253, filed Apr. 3, 2003.

### FIELD OF THE INVENTION

This invention relates to novel orally administered pharmaceutical formulations of one or more active pharmaceutical ingredients (APIs), optionally comprising salts, complexes, prodrugs and metabolites thereof, further comprising cocoa powder, to the use of one or more active pharmaceutical ingredients (APIs), optionally comprising salts, prodrugs and metabolites thereof, for the manufacturing of a medicament to be administered orally for achieving a pharmacological effect, and to methods of medical treatment of humans or animals by oral administration of one or more active pharmaceutical ingredients (APIs), optionally comprising salts, prodrugs and metabolites thereof.

### BACKGROUND

There is a need for orally administered pharmaceutical formulations of one or more active pharmaceutical ingredients (APIs) providing for a rapid, preferably intraoral uptake, such as sublingual and/or buccal uptake, and having sufficient masking of badly tasting ingredients.

### PRIOR ART

In “Development of oral acetaminophen chewable tablets with inhibited bitter taste”, Suzuki et al, International Journal of Pharmaceutics 251 (2003) 123-132 is disclosed combined use of sucrose, cocoa powder and the commercial bittermasking powder mixture Benecoat BMI-40 as corrigent against bitter taste of acetaminophen. In reality the main taste-masking effect of the Suzuki et al formulation is though achieved through the lipid matrix. But, the lipid matrix used, Witepsol H-15, is normally used for suppositories and is not suitable for oral or peroral formulations. Further, the amount of acetaminophen in the formulations of Suzuki et al is well below the general therapeutic dose. In contrast to the present formulations the formulations of Suzuki et al are not intended for intraoral uptake, but for peroral administration and subsequent uptake in the gastro-intestinal tract. A therapeutically effective unit dose of acetaminophen is too high for being administered through uptake in the oral cavity.

Chocolate, which is different from cocoa powder as such, is very rarely used as an ingredient in pharmaceutical products, hitherto only in laxatives. One example is ExLax® being chocolated laxative pieces marketed by Novartis comprising sennosides. Purex, a laxative wherein phenolphthalein was formulated with chocolate, was marketed in the 1950s.

It has now surprisingly been found that a rapid, preferably intraoral uptake of one or more APIs is achieved concomitantly with sufficient taste masking of badly tasting ingredients through use of a formulation comprising said one or more APIs and further comprising cocoa powder as taste masker, filler and texturizer.

### SUMMARY OF THE INVENTION

The present invention provides an orally administered pharmaceutical formulation of one or more APIs, optionally comprising salts, complexes, prodrugs and metabolites thereof for achieving a pharmacological effect. The administration can be to a human being or to an animal.

It is a primary objective to provide a formulation for a rapid onset through essentially intraoral uptake of one or more APIs. With “rapid onset” is herein meant that a therapeutic effect is achieved within a short period of time, preferably in less than 1 hour, more preferably in less than 30 minutes, following administration.

The administration may be accomplished without the addition of liquid. Administration without added liquid is a big advantage in all those situations where e g clean water or other suitable liquid is not available, such as on travel. Also the administration is discreet being a big advantage e g at lectures and on the theatre. Further, use of the present formulation, which should melt in the mouth rather than be swallowed, is of a great advantage to all those persons having difficulties in swallowing a traditional tablet. A particularly useful dosage form of the present invention is thus a formulation that disintegrates or melts in the mouth without need for drinking water or other fluid.

The formulation is a dosage form comprising a therapeutically effective amount of one or more APIs. It is preferred that the amount of the one or more APIs be lower than an amount causing significant side effects.

Also provided by the present invention are methods of use of formulations of the present invention for medical treatment of a human or animal subject. Other features of this invention will be in part apparent and in part pointed out hereinafter.

An object of the invention is to provide novel orally administered pharmaceutical formulations of one or more APIs comprising cocoa powder.

A second object of the invention is to provide methods for preparing said formulations.

A third object of the invention is methods for using said formulations in therapy for medical treatment of a human or animal subject.

Formulations according to the present invention should preferably melt in the oral cavity, whereby intraoral uptake of the one or more APIs is favorized.

The invention is adapted for discreet self-administration. By “discreet selfadministration” herein is meant self-adrninistration that does not draw attention to the existence of a need for therapy.

Further objects of the invention will become apparent to one skilled in the art, and still other objects will become apparent hereinafter from the specification and claims.

The main advantages provided by a formulation according to the present invention are:

1) The formulation provides for a rapid onset through essentially intraoral uptake of the one or more APIs;
2) The formulation does not require any added liquid at the time of administration;
3) The formulation provides for good taste masking;
4) The formulation does not give an immediate patient-perceived association with medicines, as do traditional tablets;
5) The formulation provides for discreet self-administration;
6) The formulation is easy to administer for persons having problems in swallowing;
7) The formulation provides for increased bioavailability due to reduced first-pass metabolism;
8) The formulation may provide for an association of pleasure.

### DETAILED DESCRIPTION OF THE INVENTION

It is the primary object of the present invention to provide orally administered cocoa-powder-containing pharmaceutical formulations useful for medical treatment.

Cocoa powder is defined as cocoa nib with some fat removed and ground into a powder. Cocoa nib is defined as cocoa beans with the shell removed. Cocoa butter is defined as fat expelled from the center (kernels or nib) of cocoa beans. Cocoa powder is prepared from roasted cocoa beans. It is a complex compound, which consists of starch, cocoa butter, amino acids, proteins, xanthines, amines, mono- and polysaccharides, phospholipids, flavonoids, pyrazines, etc.

More specifically it is the object of the invention to provide such a formulation that disintegrates and/or melts in the oral cavity with or without the aid of salivary fluid or mechanical erosion, or a combination thereof after which the formulation may show adhesiveness towards the tissues in the oral cavity.

Preferably the formulation is such that it does not require addition of liquid at the time of administration.

Optional addition of buffering agents provides for a transient change in local pH of the saliva, which facilitates uptake in the oral cavity.

It has surprisingly been found that a sufficient taste masking of badly tasting ingredients is achieved through the use of cocoa powder. The cocoa powder acts as taste masker, filler and texturizer.

A general embodiment of a formulation according to the present invention has a weight of around 200-1000 mg and has the following composition (w/w):

| | Amount | |
|---|---|---|
| Ingredient | (%) | Function |
| One or more APIs (base, prodrug, | 0.03-12 | API |
| metabolite, salt or complex) | | |
| One or more lipid ingredients | 30-60 | Lipid |
| | | ingredient |
| Cocoa powder | 8-55 | Taste masker, |
| | | filler, |
| | | texturizer |
| Water-soluble or dispersible diluents, | 0-40 | Diluent |
| preferably as fine particulate powder | | |
| One or more sweetening agents | 0.1-3 | Sweetener |
| One or more buffering agents | 0-10 | Buffer |
| One or more flavoring agents | 0-4 | Flavorant |
| One or more taste modifiers | 0-3 | Taste modifier |
| One or more emulsifiers/solubilisers | 0.3-6 | Emulsifier/ |
| | | solubiliser |
| One or more coloring agents | 0-3 | Coloring |
| | | agents |

### EXAMPLES

Below follows non-limiting examples on preparation of embodiments of the present invention.

### Example 1

A formulation, weighing around 400 mg, is prepared having the following composition (w/w):

| Ingredient | Amount (%) | Function |
|---|---|---|
| Eletriptan hydrobromide | 5.0 | API |
| Hydrogenated soybean oil | 43.55 | Lipid ingredient |
| Cocoa powder | 18.00 | Taste masker, filler, |
| | | texturizer |
| Mannitol | 12.00 | Diluent |
| Maize starch | 14.90 | Diluent |
| Aspartame | 0.15 | Sweetener |
| Acesulfame-K | 0.10 | Sweetener |
| Titanium dioxide | 2.00 | Coloring agent |
| Sodium Chloride | 0.60 | Taste modifier |
| Coffee and vanilla flavors | 3.00 | Flavoring agents |
| Soy lecithin | 0.70 | Emulsifier |

Cocoa powder may be used in a non-alkalized form and in an alkalized form. Both are useful in the present formulations. Alkalized cocoa powder is preferred when a somewhat milder taste is desirable.

A part of the hydrogenated soybean oil is melted. The solid components, i e the API if solid (eletriptan hydrobromide is solid), cocoa powder, mannitol, maize starch, aspartame, acesulfame-K, titanium dioxide, sodium chloride and the flavoring agents if solid, are added and mixed. A reduction of particle size of the solid components is performed by milling in a roll-refiner. If the solid components have already got the required particle size, e g by milling before the mixing with the fatty components, roll refining is dispensed with. After treatment in the roll-refiner the mixture is mixed with the rest of the melted fatty components or remelted, if solidified, and mixed with the rest of the melted hydrogenated soybean oil. A mixing of the melt is performed in a suitable mixer. The liquid components, i e the API if liquid (eletriptan hydrobromide is though solid and is handled as above), soy lecithin and the flavoring agents if liquid, are added. Tablets or other solid dosage forms are subsequently made using suitable techniques, such as molding, extrusion or congealing, including pastillation, when necessary after suitable preconditioning. Also other suitable manufacturing methods may be used.

### Example 2

### Preparation of a Further Embodiment

In essentially the same way as in Example 1 is manufactured a formulation with a weight of around 500 mg having the below ingredients (w/w):

| Ingredient | Amount (%) | Function |
|---|---|---|
| Terbutaline sulphate | 0.50 | Active |
| Hydrogenated soybean oil | 43.90 | Lipid ingredient |
| Cocoa powder | 18.00 | Taste masker, filler, |
| | | texturizer |
| Mannitol | 18.00 | Diluent |
| Maize starch | 13.35 | Diluent |
| Aspartame | 0.15 | Sweetener |
| Acesulfame-K | 0.10 | Sweetener |
| Titanium dioxide | 2.00 | Coloring agent |
| Mint and vanilla flavors | 3.00 | Flavoring agents |
| Soy lecithin | 1.00 | Emulsifier |

### Example 3

### Preparation of a Further Embodiment

In essentially the same way as in Example 1 is manufactured a formulation with a weight of around 300 mg having the below ingredients (w/w):

| Ingredient | Amount (%) | Function |
|---|---|---|
| Triazolam | 0.05 | Active |
| Hydrogenated soybean oil | 40.0 | Lipid ingredient |
| Cocoa powder | 37.85 | Taste masker, filler, |
| | | texturizer |
| Maize starch | 20.0 | Diluent |
| Acesulfame-K | 0.4 | Sweetener |
| Orange flavor | 1.0 | Flavor |
| Soy lecithin | 0.7 | Emulsifier |

### Example 4

### Preparation of a Further Embodiment

In essentially the same way as in Example 1 is manufactured a formulation with a weight of around 400 mg having the below ingredients (w/w):

| Ingredient | Amount (%) | Function |
|---|---|---|
| Chlorpheniramine maleate | 1.0 | Active |
| Cocoa powder | 48.7 | Taste masker, filler, |
| | | texturizer |
| Hydrogenated soybean oil | 44.0 | Lipid ingredient |
| Titanium dioxide | 2.5 | Coloring agent |
| Sodium Carbonate | 1.0 | Buffering agent |
| Aspartame | 0.2 | Sweetener |
| Acesulfame-K | 0.1 | Sweetener |
| Vanilla flavor | 1.5 | Flavoring agent |
| Soy lecithin | 1.0 | Emulsifier |

### Example 5

### Preparation of a Further Embodiment

In essentially the same way as in Example 1 is manufactured a formulation with a weight of around 600 mg having the below ingredients (w/w):

| Ingredient | Amount (%) | Function |
|---|---|---|
| Dextropropoxyphene hydrochloride | 8.0 | Active |
| Hydrogenated soybean oil | 45.0 | Lipid ingredient |
| Cocoa powder | 20.0 | Taste masker, filler, |
| | | texturizer |
| Mannitol | 19.7 | Diluent |
| Aspartame | 0.5 | Sweetener |
| Monosodium glutamate | 0.5 | Taste modifier |
| Mint flavor | 1.5 | Flavoring agent |
| Soy lecithin | 0.7 | Emulsifier |

### Example 6

### Preparation of a Further Embodiment

In essentially the same way as in Example 1 is manufactured a formulation with a weight of around 400 mg having the below ingredients (w/w):

| Ingredient | Amount (%) | Function |
|---|---|---|
| Famotidine | 2.5 | Active |
| Hydrogenated soybean oil | 42.3 | Lipid ingredient |
| Cocoa powder | 18.0 | Taste masker, filler, |
| | | texturizer |
| Mannitol | 17.5 | Diluent |
| Maize starch | 12.85 | Diluent |
| Aspartame | 0.15 | Sweetener |
| Acesulfame-K | 0.10 | Sweetener |
| Titanium dioxide | 2.0 | Coloring agent |
| Monosodium glutamate | 0.6 | Taste modifier |
| Lemon and vanilla flavors | 3.0 | Flavoring agents |
| Soy lecithin | 1.0 | Emulsifier |

### Example 7

### Preparation of a Further Embodiment

In essentially the same way as in Example 1 is manufactured a formulation with a weight of around 400 mg having the below ingredients (w/w):

| Ingredient | Amount (%) | Function |
|---|---|---|
| Metoclopramide HCl | 2.63 | Active |
| Hydrogenated soybean oil | 42.3 | Lipid ingredient |
| Cocoa powder | 17.88 | Taste masker, filler, |
| | | texturizer |
| Mannitol | 17.5 | Diluent |
| Maize starch | 12.85 | Diluent |
| Aspartame | 0.15 | Sweetener |
| Acesulfame-K | 0.10 | Sweetener |
| Titanium dioxide | 2.0 | Coloring agent |
| Monosodium glutamate | 0.6 | Taste modifier |
| Vanilla flavours | 3.0 | Flavoring agents |
| Soy lecithin | 1.0 | Emulsifier |

### Example 8

### Preparation of a Further Embodiment

In essentially the same way as in Example 1 is manufactured a formulation with a weight of around 400 mg having the below ingredients (w/w):

| Ingredient | Amount (%) | Function |
|---|---|---|
| Glyceryl trinitrate* | 0.13 | Active |
| Hydrogenated soybean oil | 44.86 | Lipid ingredient |
| Cocoa powder | 50.29 | Taste masker, filler, |
| | | texturizer |
| Aspartame | 0.62 | Sweetener |
| Mint and vanilla flavors | 3.09 | Flavoring agents |
| Lecithin | 1.03 | Emulsifier |

*as an ethanolic solution

### Example 9

### Preparation of a Further Embodiment

In essentially the same way as in Example 1 is manufactured a formulation with a weight of around 400 mg having the below ingredients (w/w):

| Ingredient | Amount (%) | Function |
|---|---|---|
| Hydralazine HCl | 6.25 | Active |
| Hydrogenated soybean oil | 40.65 | Lipid ingredient |
| Cocoa powder | 44.75 | Taste masker, filler, |
| | | texturizer |
| Aspartame | 0.6 | Sweetener |
| Mint and vanilla flavors | 1.0 | Flavoring agents |
| Lecithin | 3.75 | Emulsifier |
| Sodium carbonate | 3.0 | Buffer |

### Example 10

### Preparation of a Further Embodiment

In essentially the same way as in Example 1 is manufactured a formulation with a weight of around 400 mg having the below ingredients (w/w):

| Ingredient | Amount (%) | Function |
|---|---|---|
| Rosiglitazone maleate | 1.25 | Active |
| Hydrogenated soybean oil | 43.5 | Lipid ingredient |
| Cocoa powder | 47.25 | Taste masker, filler, |
| | | texturizer |
| Aspartame | 0.15 | Sweetener |
| Acesulfame-K | 0.10 | Sweetener |
| Vanilla flavors | 3.0 | Flavoring agents |
| Lecithin | 1.0 | Emulsifier |
| Sodium carbonate | 3.75 | Buffer |

### Example 11

### Preparation of Further Embodiments

In essentially the same way as in Example 1 are manufactured formulations with a weight from around 200 mg to around 1000 mg having the below ingredients:

| Ingredient | Amount (%) | Function |
|---|---|---|
| One or more active pharmaceutical | 0.03-12 | API |
| ingredients (APIs) as base, prodrug, | | |
| metabolite, salt or complex. | | |
| One or more lipid ingredients | 30-60 | Lipid |
| | | ingredient |
| Cocoa powder | 8-55 | Taste masker, |
| | | filler, |
| | | texturizer |
| Water-soluble or dispersible diluents, | 0-40 | Diluent |
| preferably as fine particulate powder | | |
| One or more sweetening agents | 0.1-3 | Sweetener |
| One or more buffering agents | 0-10 | Buffer |
| One or more flavoring agents | 0-4 | Flavor |
| One or more taste modifiers | 0-3 | Taste modifier |
| One or more emulsifiers/solubilizers | 0.3-6 | Emulsifier |
| One or more coloring agents | 0-3 | Coloring |
| | | agents |

The above one or more active pharmaceutical ingredients (APIs) is/are selected from APIs suitable for intraoral uptake, preferred, but non-limiting examples of which are
the antiinflammatory agents diclofenac, ketorolac, indometacin, tomoxicam, piroxicam, tenoxicam, ketoprofen, celecoxib and roficoxib;
the muscle relaxants orphenadrine and baclofen;
the drugs affecting bone mineralization alendronic acid and risedronic acid;
the analgesics propoxyphene, buprenorfin, ketobenidon, hydromorphone, tramadol and morphine
the antimigraine preparations dihydroergotamine, ergotamine, eletriptan, naratriptan, rizatriptan, sumatriptan and zolmitriptan;
the anti-Parkinson drugs pramipexole, ropinirole and selegiline;
the anxiolytics alprazolam, diazepam, lorazepam and oxazepam;
the hypnotics flunitrazepam, midazolam, nitrazepam, triazolam, zaleplone, zopiclone, zolpiderm, clomethiazole and propiomazine;
the psychostimulant caffeine;
the drugs against substance dependence bupropione, lobeline, nicotine, naltrexone and methadone;
the gastric ulcer remedy famotidine;
the antispasmodic hyoscyamine;
the antiemetics metoclopramide, ondansetron, scopolamine, hyoscine, perfenazine, procloperazine and haloperidol:
the antidiabetic agent rosiglitazone;
the cardiovascular agents etilefrin, glyceryl trinitrate, isosorbide dinitrate and isosorbide mononitrate;
the antihypertensive agent hydralazine;
the diuretics furosemide and amiloride;
the beta-receptor blocking agents propranolol and timolol;
the calcium channel blocker amlodipine;
the ACE-inhibitors kaptopril, lisinopril and fosinopril;
the serum lipid reducing agent simvastatin;
the antipsoriatic acitretin;
the antiasthmatic terbutaline;
the antitussives codeine and noscapine, and
the antihistamines clemastine, chlorpheniramine, cyproheptadine, loratadine and acrivastine.

### Example 12

### Preparation of Alternative Embodiments

Useful embodiments are obtained by exchanging some of the excipients in the embodiments of the above examples 1-11 for equivalently functioning alternative compounds.

The cocoa powder may be used in its non-alkalized form, its alkalized form or in a mixture thereof.

The diluents may be selected from one or more of the compounds sucrose, fructose, glucose, galactose, lactose, maltose, invert sugar, a pharmaceutically acceptable polyol such as xylitol, sorbitol, maltitol, mannitol, isomalt and glycerol, or polydextrose, or starch, or any mixture thereof, but only to such an extent that the taste-masking effect of the cocoa-powder remains sufficient.

The lipid ingredient, being fatty components, may be chosen from one or more of the following compounds:
cocoa butter and cocoa butter alternatives, including cocoa butter equivalents (CBE), cocoa butter substitutes (CBS), cocoa butter replacers (CBR) and cocoa butter improvers (CBI),
coconut, palmkernel oil and other similar oils characterized by being predominantly based on lauric and myristic acids,
palm oil, shea butter, karite butter, illipe butter, mango kernel oil, sal fat and other similar fats characterized by being predominantly based on palmitic, oleic and stearic acids,
corn oil, sunflower oil, hybrid sunflower oil, soybean oil, rapeseed oil, canola oil, olive oil, ricebran oil, cottonseed oil, arachis (peanut, groundnut) oil and other oils characterized by being predominantly based on oleic, linoleic and linolenic acids and hydrogenated to a suitable melting point,
fish oil, tallow, lard, butterfat and other animal derived fats, and
synthetic fats, reesterified fats, hard fats obtained by a chemical reaction of fatty acids with glycerol using no, acidic, alkaline or enzymatic catalysis, whereby said compound/s is/are used as a single component or mixed with each other, being either crude or refined using physical or alkaline refining, or being sub-jected to further processing including catalytic hydrogenation, interesterification, transesterification and fractionation.

The optional buffering agent/s may be selected from one or more of carbonates, bicarbonates, acetates, gluconates, glycerophosphates, phosphates or glycinates of sodium, potassium or ammonium, or mixtures thereof. Most phosphates are though less suitable because their taste usually is disagreeable and difficult to mask. Addition of buffering agents/s may increase the uptake through the mucosa in the oral cavity.

The sweetener may selected from one or more artificial sweeteners, such as sucrose, aspartame, acesulfame potassium, saccharine, sodium saccharine, cyclamate, glycyrrhizine, thaumatin (talin), sucralose, dihydrochalcone (neohesperidin dihydrochalcone), alitame, miraculin (miracle fruit), monellin (serendipity berry), stevside and/or salts thereof.

The emulsifier/solubiliser is preferably soy lecithin and/or egg lecithin, but may be exchanged for
a nonionic surfactant, such as poloxamer, polyoxyethylene alkyl ether, polyoxyethylene castor oil derivative, polyoxyethylene sorbitan fatty acid ester, monoglyceride, diglyceride and esther thereof, polyoxyethylene stearate, polyglycerolester of fatty acids, including polyglycerolpolyricinoleic acid (PGPR), sorbitan fatty acid ester,
an anionic surfactant, such as fatty acid, soap of fatty acid, lactylate, especially sodium and/or calcium stearoyllactylate, sodium lauryl sulfate and latanol,
a zwitterionic surfactant, such as zwitterionic phospholipid, such as phosphatidylcholine and phosphatidylethanolamine, or mixtures, fractions or derivatives thereof or with lecithin.

The taste modifier is preferably selected from sodium chloride, monosodium glutamate and ammonium glycyrrhizinate.

The coloring agent is preferably selected from titanium dioxide, iron oxides and aluminum lakes.

Formulations according to the present inventions primarily constitute meltable and/or suckable oral tablets, but also include other suitable dosage forms for intraoral administration such as buccal patches, buccal pastes and buccal sprays.

Further, the present invention encompasses administration of the captioned formulations via the oral route concomitantly with administration APIs via one or more other routes, such as through transdermal administration, peroral administration, administration by inhalation, administration by creams, salves and vagitories, and/or administration by injection.

1. An orally administered pharmaceutical formulation comprising at least one active pharmaceutical ingredient capable of intraoral uptake and cocoa powder, wherein the active pharmaceutical ingredient is selected from the group consisting of selegiline, propranolol, and chlorpheniramine.

2. The formulation according to claim 1 further comprising at least one lipid.

3. The formulation according to claim 2 wherein said lipid ingredient is selected from the group consisting of cocoa butter, cocoa butter equivalents, cocoa butter substitutes, cocoa butter replacers, cocoa butter improvers, coconut, palmkernel oil, palm oil, shea butter, karite butter, illipe butter, mango kernel oil, sal fat, corn oil, sunflower oil, hybrid sunflower oil, soybean oil, rapeseed oil, canola oil, olive oil, ricebran oil, cottonseed oil, arachis (peanut, groundnut) oil, fish oil, tallow, lard, butterfat and mixtures thereof.

4. The formulation according to claim 3, wherein said lipid comprises a member selected from the group consisting of cocoa butter equivalents, cocoa butter substitutes and cocoa butter replacers.

5. The formulation according to claim 1, further comprising at least one buffering agent.

6. The formulation according to claim 5, wherein said buffering agent is selected from the group consisting of carbonates, bicarbonates, acetates, gluconates, glycerophosphates, phosphates or glycinates of sodium, potassium or ammonium, and mixtures thereof.

7. The formulation according to claim 1, further comprising at least one sweetener.

8. The formulation according to claim 7, wherein said at least one sweetener is selected from the group consisting of sucrose, aspartame, acesulfame potassium, saccharine, sodium saccharine, cyclamate, glycyrrhizine, thaumatin, sucralose, dihydrochalcon, neohesperidin dihydrochalcone, alitame, miraculin, monellin, stevside and salts and mixtures thereof.

9. The formulation according to claim 1, further comprising at least one emulsifier.

10. The formulation according to claim 9, wherein said emulsifier is selected from the group consisting of lecithin, poloxamer, polyoxyethylene alkyl ether, polyoxyethylene castor oil derivative, polyoxyethylene sorbitan fatty acid ester, monoglyceride, diglyceride and esther thereof, polyoxyethylene stearate, polyglycerolester of fatty acids sorbitan fatty acid ester, fatty acid, soap of fatty acid, lactylate, especially sodium and/or calcium stearoyllactylate, sodium lauryl sulfate, latanol, phosphatidylcholine, phosphatidylethanolamine, and mixtures thereof.

11. The formulation according to claim 10, wherein said one or more emulsifier is lecithin.

12. The formulation according to claim 1, further comprising a taste modifier, selected from the group consisting of sodium chloride, monosodium glutamate and ammonium glycyrrhizinate.

13. The formulation according to claim 1, further comprising a coloring agent, selected from the group consisting of titanium dioxide, iron oxides and aluminum lakes.
